(19) United States
(12) Plant Patent Application Publication
(10) Pub. No.: US 2019/0320572 P1
(43) Pub. Date: Oct. 17, 2019

(54) Echeveria plant named 'TSAECH1820'

(50) ;

(71) Applicant: Tosjami B.V., Heerhugowaard (NL)

(72) Inventor: Gerard van Langen, Heerhugowaard (NL)

(73) Assignee: Tosjami B.V., Heerhugowaard (NL)

(21) Appl. No.: 16/501,054

(22) Filed: Feb. 14, 2019

(30) Foreign Application Priority Data
Apr. 13, 2018 (QZ) 2018/1026

(51) Int. Cl. A01H 6/00

(52) U.S. Cl. PLT/373

(57) ABSTRACT

A new and distinct Echeveria plant named ‘TSAECH1820’ which is characterized by improved plant vigor, an abundance of succulent foliage tightly held in a compact stemless rosette, heavily glaucous foliage at and near the center of the rosette which generally appears to be a mixture of blue-gray and greyed-green, foliage towards the outer whorls of the rosette which becomes decreasingly glaucous and therefore progressively darker, closer to greyed-green, foliage which is conspicuously margined and tipped pink, and the stability of these characteristics from generation to generation.

### CROSS REFERENCE TO RELATED APPLICATIONS

This application claims priority to the Community Plant Variety Rights application number 2018/1026, filed Apr. 13, 2018, which is herein incorporated by reference.

Latin name of the genus and species: The Latin name of the genus and species of the novel variety disclosed herein is Echeveria hybrid.

Variety denomination: The inventive variety of Echeveria disclosed herein has been given the variety denomination ‘TSAECH1820’.

### BACKGROUND OF THE INVENTION

Parentage: ‘TSAECH1820’ is a seedling selection resulting from the controlled pollination of an unnamed and unpatented Echeveria pulidonis plant, the seed parent, with an unnamed and unpatented Echeveria laui plant, the pollen parent. The crossing was made by the inventor in the spring of 2014 at a commercial greenhouse in Heerhugowaard, the Netherlands. In the summer of 2015, one seedling resulting from the cross of 2014 was observed to exhibit unique growth and foliage characteristics and was isolated for further evaluation in order to confirm the distinctness and stability of the characteristics first observed. Upon confirmation of distinctness and stability, ‘TSAECH1820’ was selected for commercialization.

Asexual Reproduction: Asexual reproduction of the new cultivar ‘TSAECH1820’, by way of rooting leaf cuttings, was first initiated in the spring of 2016 at the inventor's commercial greenhouse in Heerhugowaard, the Netherlands. Through four subsequent generations, the unique features of this cultivar have proven to be stable and true to type.

### SUMMARY OF THE INVENTION

The cultivar ‘TSAECH1820’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘TSAECH1820’. These characteristics in combination distinguish ‘TSAECH1820’ as a new and distinct Echeveria cultivar:
1. ‘TSAECH1820’ exhibits improved plant vigor, with lower mortality rates under adverse climatic conditions; and
2. ‘TSAECH1820’ exhibits an abundance of succulent foliage tightly held in a compact stemless rosette; and
3. ‘TSAECH1820’ exhibits heavily glaucous foliage at and near the center of the rosette, the color of which has a general appearance of a mixture between blue-grey and greyed-green; and
4. ‘TSAECH1820’ exhibits foliage towards the outer whorls of the rosette which becomes decreasingly glaucous and therefore progressively darker, closer to greyed-green; and
5. ‘TSAECH1820’ exhibits foliage which is conspicuously margined and tipped pink.

### BRIEF DESCRIPTION OF THE FIGURES

FIG. 1 illustrates, as nearly true as it is reasonably possible to make the same in color photographs of this type, an exemplary plant of ‘TSAECH1820’ grown in a commercial greenhouse in Heerhugowaard, the Netherlands. This plant is approximately 12 months old, shown planted in a 12 cm container.

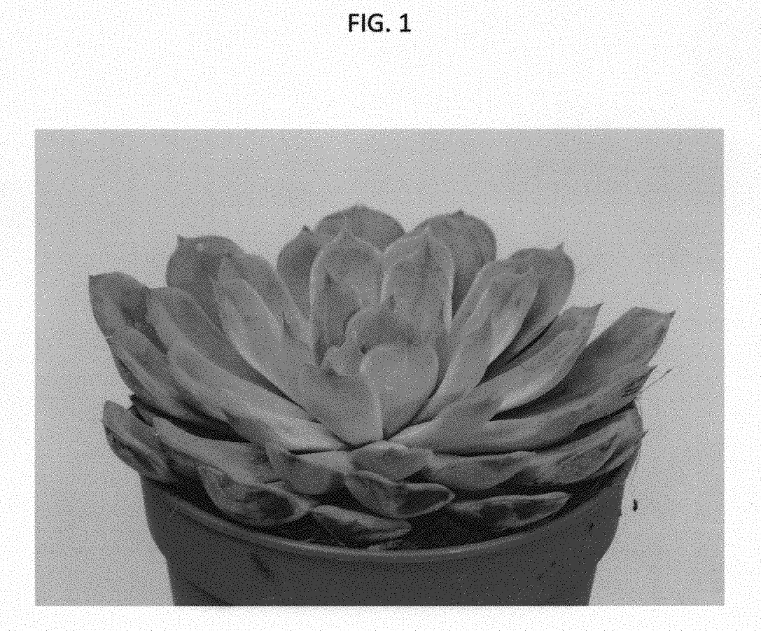

FIG. 2 illustrates, as nearly true as it is reasonably possible to make the same in color photographs of this type, the typical foliage arrangement of ‘TSAECH1820.

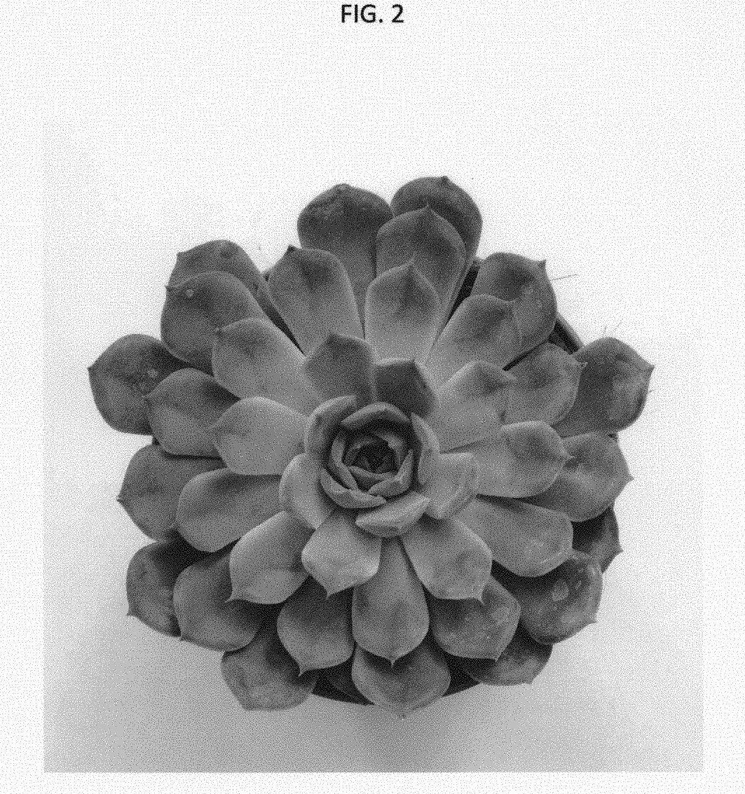

FIG. 3 illustrates, as nearly true as it is reasonably possible to make the same in color photographs of this type, the adaxial surface of the juvenile and mature foliage of ‘TSAECH1820’.

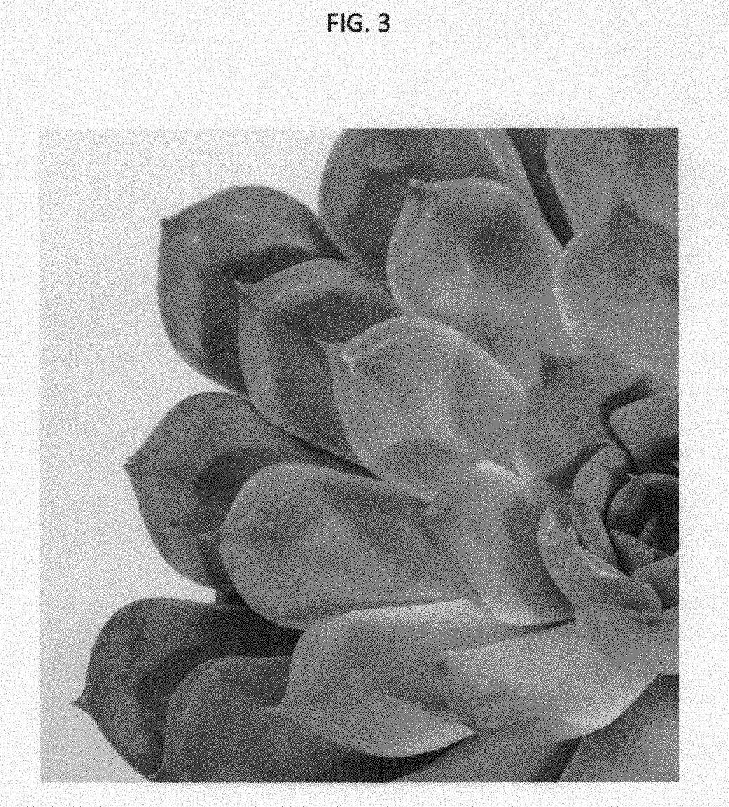

FIG. 4 illustrates, as nearly true as it is reasonably possible to make the same in color photographs of this type, the abaxial surface of the mature foliage of ‘TSAECH1820’.

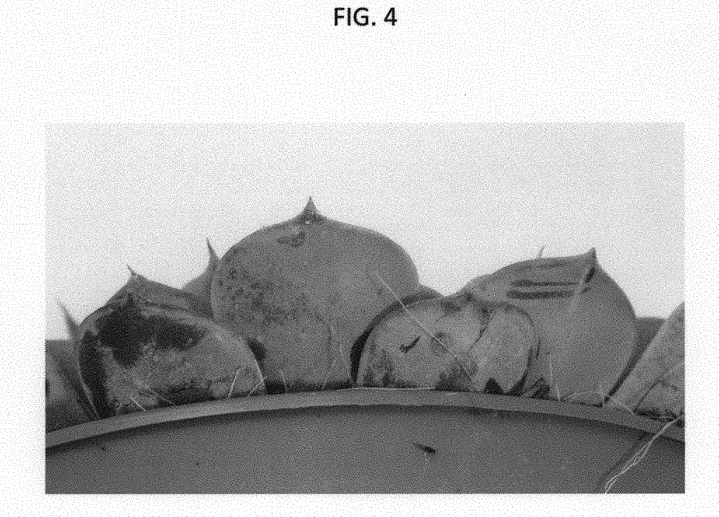

### BOTANICAL DESCRIPTION OF THE PLANT

The following observations and measurements made in December of 2018 describe averages from a sample set of six specimens of 12 months old ‘TSAECH1820’ plants grown in 12 cm nursery containers at commercial greenhouse in Heerhugowaard, the Netherlands. Plants were produced using conventional greenhouse production protocols for Echeveria plants which consisted of minimal irrigation and fertilizer applications, and chemical pest and disease control measures against mealy bug and Botrytis as required. Plants were grown under approximately 50 percent shade after propagation and later exposed to full sun once they began to mature. No photoperiodic treatments or artificial light was given to the plants.

Those skilled in the art will appreciate that certain characteristics will vary with older or, conversely, with younger plants. ‘TSAECH1820’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climatic and cultural conditions. Color notations are based on The Royal Horticultural Society Colour Chart, The Royal Horticultural Society, London, 2015 (sixth edition).

A botanical description of ‘TSAECH1820’ and comparisons with the parent plants and closest known comparator are provided below.
Plant description:
   Growth habit.—Succulent perennial with foliage growing in a stemless basal rosette.
   Plant shape.—Flattened.
   Height from soil level to top of foliar plane.—4.7 cm.
   Plant spread.—Average of 14.0 cm.
   Growth rate.—Moderately fast growing.
   Plant vigor.—Moderately vigorous.
   Propagation.—Type — Leaf cuttings. Time to initiate rooting — Approximately 21 days at 18 degrees Celsius. Crop time — Approximately 35 weeks to produce a marketable plant in a 10.5 cm container.
   Disease and pest resistance or susceptibility.—Neither resistance nor susceptibility to typical Echeveria pests and diseases has been observed.
   Environmental tolerances.—Adapt to, at least, USDA Zones 10 to 12 and temperatures as high as 40 degrees Celsius; moderate tolerance to rain yet drought tolerant once established; high tolerance to wind.
Root system:
   General.—Fine, well-branched fibrous roots.
Foliage:
   Arrangement.—Rosette.
   Division.—Simple.
   Attachment.—Sessile.
   Quantity.—Approximately 50 leaves per rosette.
   Shape.—Obovate to broad oblanceolate.
   Dimensions.—5.8 cm long, 2.4 cm wide, and 0.7 cm thick, on average.
   Aspect.—Slightly concave and very slightly curved upwards.
   Attitude.—Juvenile foliage at the center of the rosette is held upward and outward; foliage becomes progressively more relaxed towards the outer whorls of mature foliage, at an average angle of 20 degrees to horizontal.
   Apex.—Apiculate.
   Base.—Long cuneate.
   Margin.—Entire; not undulated.
   Pubescence, texture and luster of the adaxial surface.—Glabrous, smooth, and matte; moderately to heavily glaucous.
   Pubescence, texture and luster of the abaxial surface.—Glabrous, smooth, and matte; moderately to heavily glaucous.
   Color.—Juvenile foliage, adaxial surface — Nearest to in between greyed-green and yellow-green, RHS 191A and 148B, and fading to a lighter shade of yellow-green towards the base, nearest to RHS N148D; distally margined and tipped greyed-red, nearest to RHS 182B; the epicuticular wax covering the leaf surface is nearest to in between blue-green and greyed-green, RHS 122D and 188C. Juvenile foliage, abaxial surface — Yellow-green, nearest to RHS 148B, and fading to a lighter shade of yellow-green towards the base, nearest to RHS N148D; distally margined greyed-red, nearest to RHS 181C, and tipped a darker shade of greyed-red, nearest to RHS 182A; the epicuticular wax covering the leaf surface is nearest to in between blue-green and greyed-green, RHS 122C and 188D. Mature foliage, adaxial surface — Yellow-green, nearest to RHS 148A, and fading to a lighter shade of yellow-green towards the base, nearest to RHS 146C; margined greyed-red, nearest to a mixture of RHS 181B and 181C; the epicuticular wax covering the leaf surface is nearest to in between blue-green and greyed-green, RHS 122D and 188C. Mature foliage, abaxial surface — Yellow-green, nearest to RHS 147C, and heavily suffused with greyed-red towards the apex, nearest to a mixture of RHS 178A and 178B; margined greyed-red, nearest to a mixture of RHS 181B and 181C; the epicuticular wax covering the leaf surface is nearest to in between blue-green and greyed-green, RHS 122C and 188D.
   Venation.—No venation is visible.
   Petiole.—No petiole; leaves are sessile.

### INFLORESCENCE

No flowering has been observed to date.

### COMPARISONS WITH THE PARENT PLANT AND CLOSEST KNOWN COMPARATOR

Plants of the new cultivar ‘TSAECH1820’ differ from the seed parent, an unnamed Echeveria pulidonis plant (not patented), in the following characteristics described in Table 1 below.

TABLE 1

| Characteristic | ‘TSAECH1820’ | Seed parent |
|---|---|---|
| General coloration | A mixture of blue-gray and | Blue-green. |
| of the foliage. | greyed-green, although | |
| | darker than the parent. | |
| General coloration | Pink. | Red. |
| of the margins. | | |

Plants of the new cultivar ‘TSAECH1820’ differ from the pollen parent, an unnamed Echeveria laui plant (not patented), in the following characteristics described in Table 2 below.

TABLE 2

| Characteristic | ‘TSAECH1820’ | Pollen parent |
|---|---|---|
| Plant vigor. | More vigorous than the | Less vigorous than |
| | pollen parent; lower | ‘TSAECH1820’; higher |
| | mortality rate under | mortality rate under |
| | adverse climatic | adverse climatic |
| | conditions. | conditions. |
| Abundance of | More abundant than the | Less abundant than |
| foliage. | pollen parent. | ‘TSAECH1820’. |
| Foliage | Longer and more narrow | Shorter and broader than |
| dimensions. | than the pollen parent. | ‘TSAECH1820’. |
| General coloration | Generally appearing as a | Generally appearing as |
| of the foliage. | mixture of blue-gray and | verily pale blue-gray to |
| | greyed-green, although | nearly white. |
| | darker than the parent. | |

### COMPARISONS WITH THE CLOSEST KNOWN COMPARATOR

Plants of the new cultivar ‘TSAECH1820’ differ from the closest known commercial comparator, Echeveria sp. ‘Orion’ (U.S. Pat. No. 26,228), in the following characteristics described in Table 3 below.

TABLE 3

| Characteristic | ‘TSAECH1820’ | ‘Orion’ |
|---|---|---|
| General | Generally appearing as a | Generally appearing as a |
| coloration | mixture of blue-gray and | mixture of blue-gray and |
| of the foliage. | greyed-green, although | greyed-green, although |
| | darker than ‘Orion’. | lighter than ‘TSAECH1820’. |
| General | Generally appearing as a | Generally appearing as a |
| coloration | darker shade of pink. | lighter shade of pink. |
| of the margins. | | |

That which is claimed is:

1. A new and distinct variety of Echeveria plant named ‘TSAECH1820’, substantially as described and illustrated herein.
